(19) United States
(12) Patent Application Publication
(10) Pub. No.: US 2024/0270683 A1
(43) Pub. Date: Aug. 15, 2024

(54) A PROCESS FOR PREPARATION OF AN INTERMEDIATE OF L-GLUFOSINATE

(71) Applicants: Chandrasekhar Dayal MUDALIAR, Mumbai (IN); Ashishkumar Ravindra MISHRA, Maharashtra, Thane (IN)

(72) Inventors: Chandrasekhar Dayal MUDALIAR, Mumbai (IN); Ashishkumar Ravindra MISHRA, Maharashtra, Thane (IN)

(21) Appl. No.: 18/565,177

(22) PCT Filed: Jun. 15, 2022
(86) PCT No.: PCT/IN2022/050546

§ 371 Date: Nov. 29, 2023

(30) Foreign Application Priority Data
Jun. 17, 2021 (IN) 202121027188

(51) Int. Cl. C07C 271/22; C07F 9/30

(52) U.S. Cl. CPC C07C 271/22; C07F 9/301

(57) ABSTRACT

The present invention provides a process for preparation of compound of formula (I), a key intermediate in synthesis of L-glufosinate or its salt. wherein P² is an amino-protecting group; R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and X is a halogen or hydroxyl group.

### FIELD OF THE INVENTION

The present invention relates to a process for the preparation of compound of formula (I) which is a key intermediate in the synthesis of L-glufosinate or its salts. The present invention further relates to a process for the preparation of L-glufosinate or its salts using compound of formula (I).

### BACKGROUND OF THE INVENTION

Glufosinate is non-selective herbicide belonging to the group of organophosphate and has been widely used around the world. It is generally used in the form of ammonium salt for total vegetation control and to control growth of weeds and grasses. Glufosinate is used as a racemic mixture of L- & D-glufosinate. However, it is well known that L-glufosinate i.e. (S)-2-amino-4-(hydroxy(methyl)phosphonoyl)butanoic acid) is much more potent than D-glufosinate.

There are various methods for preparation of L-glufosinate, and one of the convenient methods for the preparation of L-glufosinate proceeds through formation of compound of formula (I). The methods for the preparation of compound of formula (I) known in the prior art are lengthy, tedious and proceed through isolation of multiple intermediates thereby effecting the overall yield indirectly. Another problem associated with the known processes is that the intermediates formed during process are sensitive and tend to undergo reversible reactions leading to formation of by-products or convert back to starting materials. This impacts the purity of the final product and requires multiple purification steps. To overcome these problems associated with prior art processes, the inventors of present invention have developed a shorter, economical and scalable route for synthesis of L-glufosinate and its key intermediate. Certain alternatives are available in the early stages of this route, which is indeed advantageous since it opens the way to use different reaction strategies. All these alternative routes however lead to formation of same key intermediate i.e. compound of formula (I).

### OBJECT OF THE INVENTION

One object of the present invention is to provide a process for preparation of a compound of formula (I).

Another object of present invention is to provide simple, economical and scalable process for preparation of a compound of formula (I).

Yet another object of the present invention is to provide a process for preparation of L-glufosinate or its salts, using the compound of formula (I) prepared according to present invention.

### SUMMARY OF THE INVENTION

According to an aspect, there is provided a process for preparation of compound of formula (I),

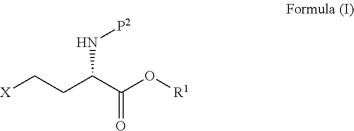

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group;
from a compound of formula (II),

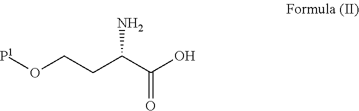

wherein P¹ is hydroxyl-protecting group;
comprising steps of protection of amino group, deprotection of hydroxyl group and halogenation.

According to an aspect of the present invention, there is provided a process for preparation of compound of formula (I),

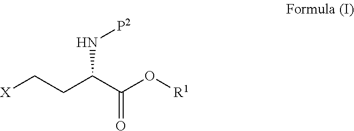

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
comprising steps of:
   i) protecting the amine group of compound of formula (II) or its salt to obtain compound of formula (III),

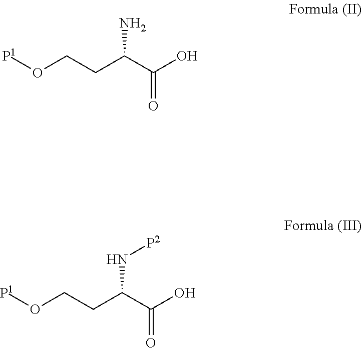

wherein P¹ is hydroxyl-protecting group; and P² is an amino-protecting group; and
ii) converting the compound of formula (III) to compound of formula (I),
wherein the step i) is carried out by maintaining pH in the range of 4 to 7.

According to an aspect of the present invention, there is provided a compound of formula (III)

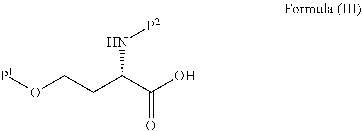

wherein P¹ and P² are defined above.

According to another aspect of the present invention, there is provided a process for preparation of compound of formula (I),

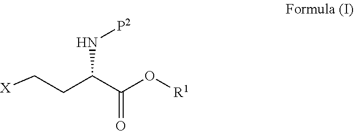

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
comprising steps of:
   a) reacting a compound of formula (II) or its salts with an acid to obtain compound of formula (V) or its salt;

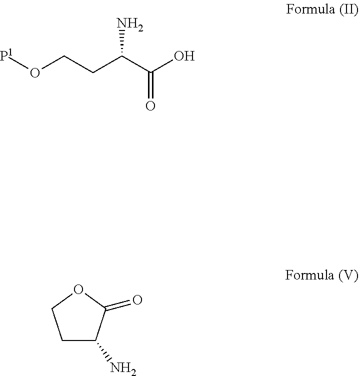

b) protecting the amine group of compound of formula (V) or its salt to obtain compound of formula (VI),

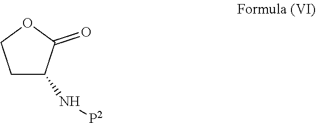

wherein P² is same as defined above; and
c) treating the compound of formula (VI) with a halogenating agent wherein the step b) is carried out by maintaining pH in the range of 4 to 7.

According to yet another aspect of the present invention, there is provided a process for preparation of L-glufosinate or its salt, comprising reacting a compound of formula (I) obtained by one of the processes described above, with a phosphorous containing compound and subsequently converting to L-glufosinate or its salt.

### DETAILED DESCRIPTION OF THE INVENTION

Those skilled in art will be aware that invention described herein is subject to variations and modifications other than those specifically described. It is to be understood that the invention described herein includes all such variations and modifications. The invention also includes all such steps, features, compositions and methods referred to or indicated in this specification, individually or collectively, and any and all combinations of any two or more said steps or features.

### Definitions

For convenience, before providing further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The terms used herein are defined as follows.

As used in the specification and the claims, the singular forms “a”, “an”, and “the” include plural referents unless the context clearly dictates otherwise.

The present disclosure is not to be limited in scope by the specific embodiments described herein, which are intended for the purposes of exemplification only.

The term “room temperature” unless stated otherwise, essentially means temperature in range of 20-35° C.

The term “purity” means purity as determined by HPLC (“High Pressure Liquid Chromatography”).

The term “about” shall be interpreted to mean “approximately” or “reasonably close to” and any statistically insignificant variations therefrom. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or ±5% of the stated value. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

As used herein, the terms “comprising” “including,” “having,” “containing,” “involving,” and the like are to be understood to be open-ended, i.e., to mean including but not limited to.

The terms “preferred” and “preferably” refer to embodiments of the invention that may afford certain benefits, under certain circumstances. In an embodiment, the aspects and embodiments described herein shall also be interpreted to replace the clause “comprising” with either “consisting of” or with “consisting essentially of” or with “consisting substantially of”.

The term “amino-protecting group” as used herein refers to a substituent that protects an amino functionality against undesirable reactions during synthetic procedures. Amino-protecting groups are typically selected from acyl group such as C(═O)Y, wherein Y can be OR or R, wherein R is substituted or unsubstituted C₁ to C₁₀ alkyl group, a substituted or unsubstituted C₁ to C₁₀ alkenyl group, a substituted or unsubstituted C₁ to C₁₀ alkynyl group, a substituted or unsubstituted C₃ to C₁₀ cycloalkyl group, a substituted or unsubstituted C₆ to C₂₀ aryl group, or a substituted or unsubstituted C₂ to C₁₀heteroaryl group; or it can be urea, urethane, nitroso, nitro, sulphenyl, sulphonyl, sulfonic acid, or trialkylsilyl. Examples include acetyl, carbobenzyloxy (also benzyloxycarbonyl or carbobenzoxy), formyl, t-butyloxycarbonyl, fluorenylmethyloxycarbonyl, 2-nitrophenylsulfenyl, methanesulfonyl, p-toluenesulfonyl, p-nitro phenyl sulfonyl group and the like.

The term “hydroxyl-protecting group” “as used herein refers to a substituent that protects a hydroxyl functionality against undesirable reactions during synthetic procedures. Hydroxyl-protecting groups are typically selected from a substituted or unsubstituted C₁ to C₁₅ acyl group derived from mono or dicarboxylic acid such as formyl group, acetyl group, fumaryl group, maleyl group, succinyl group, benzoyl group; a —COR^{a}, wherein Ra is selected from substituted or unsubstituted C₁ to C₁₅ alkyl, C₁ to C₁₅ alkenyl, C₁ to C₁₅ alkyne, C₆ to C₂₀ aryl and aralkyl groups; a —SO₃H group; —SO₂R^{b} wherein R^{b} is selected from substituted or unsubstituted C₁ to C₁₅ alkyl, aryl and aralkyl groups; silyl group —SiR^{c}R^{d}R^{e} wherein R^{c}, R^{d}, R^{e} may be the same or different and are selected from the group consisting of C₁-C₆ alkyl, aryl and aralkyl groups, and —(CH₂)ₙO—R′ wherein R′ is selected from the group consisting of C₁-C₆ alkyl, aryl and aralkyl groups and a tetrahydropyranyl; 0-trifluoromethyl sulfonyl (triflates); 0-trifluoromethyl acetyl.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

“Alkyl” as used herein means a straight or branched chain saturated aliphatic hydrocarbon having the specified number of carbon atoms, specifically 1 to 12 carbon atoms, more specifically 1 to 8 carbon atoms.

“Halogen” as used herein means a fluorine, chlorine, bromine or iodine atoms.

“Alkenyl” as used herein means a straight or branched chain unsaturated aliphatic hydrocarbon with carbon-carbon double bond having the specified number of carbon atoms, specifically 2 to 15 carbon atoms.

“Alkynyl” refers to an alkyl group containing at least one carbon-carbon triple bond and having from 2 to 15 carbon atoms. The term “alkynyl” also includes those groups having one triple bond and one double bond.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, and the likes

“Aryl” refers to an aromatic carbocyclic group having a single ring (e.g. monocyclic) or multiple rings (e.g. bicyclic or tricyclic) including fused systems. Examples of aryl groups include phenyl, naphthyl, and the likes.

As used herein, the term “L-glufosinate” refers to the L-isomer of glufosinate, a salt and an ester thereof. The L-isomer of glufosinate is a structural analogue of glutamate and, therefore, is a competitive inhibitor of the enzyme glutamine synthetase (GS) of bacteria and plants. The L-enantiomer of glufosinate acts by inhibition of glutamine synthetase thereby causing accumulation of toxic levels of ammonium ion and indirectly stopping photosynthesis. It is also known as phosphinothricin or (S)-2-amino-4-(hydroxy(methyl)phosphonoyl)butanoic acid. The term can generically refer to any form of L-glufosinate such as solvates, hydrates, esters, anhydrous form, polymorph forms, pseudo polymorph forms, amorphous form or mixture thereof, and sodium, potassium or ammonium salts.

The term “L-glufosinate” shall be interpreted to mean L-glufosinate or its salts.

The salts of L-glufosinate such as monosodium salt, disodium salt, monopotassium salt, dipotassium salt, calcium salt, ammonium salt, —NH₃(CH₃)⁺ salt, —NH₂(CH₃)₂⁺ salt, —NH(CH₃)³⁺ salt, —NH(CH₃)₂(C₂H₄OH)⁺ salt, and —NH₂(CH₃)(C₂H₄OH)⁺ salt are included in the definition. The agronomically acceptable salts include L-glufosinate-ammonium, L-glufosinate-sodium, and L-glufosinate-potassium. The term may also refer to an isomeric (racemic) mixture of L-glufosinate, D-glufosinate and salts thereof, wherein the content of L-glufosinate in the mixture is 70% or greater, preferably 80% or greater and more preferably 90% or greater. Typically, the ratio of L-glufosinate: D-glufosinate can be in the range from about 90:10 to about 99.9:0.1, preferably from about 95:5 to about 99.9:0.1.

According to an aspect of the present invention, there is provided a process for preparation of compound of formula (I),

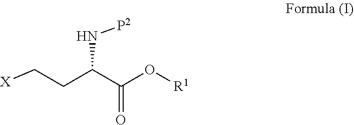

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
wherein said process for the preparation of the compound of formula (I) proceeds via a compound of formula (II),

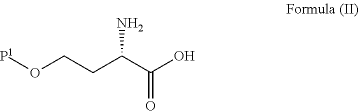

wherein P¹ is hydroxyl-protecting group.

According to an aspect of the present invention, there is provided a process for preparation of compound of formula (I),

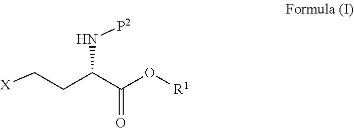

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
from a compound of formula (II),

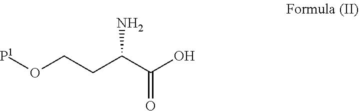

wherein P¹ is hydroxyl-protecting group,
comprising steps of protection of amino group, deprotection of hydroxyl group and halogenation.

In an embodiment, steps of protection of amino group and deprotection of hydroxyl group can take place in any order.

According to an aspect of the present invention, there is provided a process for preparation of compound of formula (I),

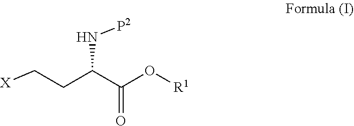

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
comprising steps of:
   i) protecting the amine group of compound of formula (II) or its salt to obtain compound of formula (III),

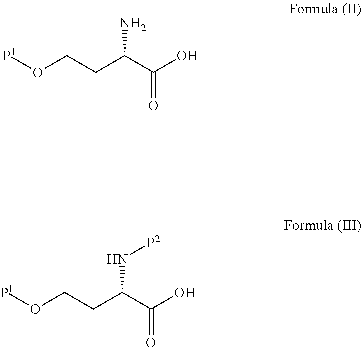

wherein P¹ is hydroxy-protecting group; and P² is an amino-protecting group; and
ii) converting compound of formula (III) to compound of formula (I),
wherein the step i) is carried out by maintaining pH in the range of 4 to 7.

According to an aspect there is provided a process for preparation of compound of formula (III) comprising
protecting the amine group of a compound of formula (II) or its salt to obtain a compound of formula (III),

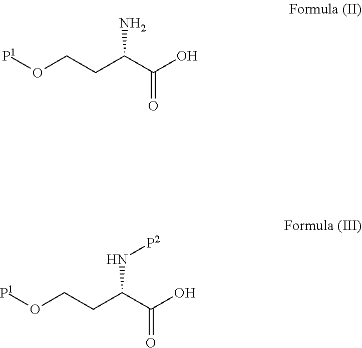

wherein P¹ is hydroxy-protecting group; and P² is an amino-protecting group.

In an embodiment the process for obtaining compound of formula (III) from a compound of formula (II) or its salt is carried out at temperature in the range of −10 to 50° C. The process is carried out for a period in the range of 0.5 to 20 hours.

In an embodiment, P¹ is hydroxyl-protecting group selected from group comprising of substituted or unsubstituted C₁ to C₁₅ acyl group derived from mono or dicarboxylic acid; a —COR^{a}, wherein Ra is selected from substituted or unsubstituted C₁ to C₁₅ alkyl, C₁ to C₁₅ alkenyl, C₁ to C₁₅ alkyne, C₆ to C₂₀ aryl and aralkyl groups; a —SO₃H group; —SO₂R^{b} wherein R^{b} is selected from substituted or unsubstituted C₁ to C₁₅ alkyl, aryl and aralkyl groups; silyl group —SiR^{c}R^{d}R^{e} wherein R^{c}, R^{d}, R^{e} may be the same or different and are selected from the group consisting of C₁-C₆ alkyl, aryl and aralkyl groups, —(CH₂)ₙO—R′ wherein R′ is selected from the group consisting of C₁-C₆ alkyl, aryl and aralkyl groups or a tetrahydropyranyl; O-trifluoromethyl sulfonyl (triflates); or O-trifluoromethyl acetyl.

According to an embodiment, the P¹ is selected from the group comprising of an acyl group derived from substituted or unsubstituted C₁ to C₁₅ mono or dicarboxylic acid. In an embodiment the P¹ substitution of compound of formula (II) is selected from, but not limited to, an acyl group derived from substituted or unsubstituted C₁ to C₁₅ mono or dicarboxylic acid such as formyl group, acetyl group, fumaryl group, maleyl group, succinyl group or benzoyl group.

According to an embodiment, P² is an amino-protecting group selected from group comprising of acyl group such as C(═O)Y, wherein Y can be OR or R, wherein R is substituted or unsubstituted C₁ to C₁₀ alkyl group, a substituted or unsubstituted C₁ to C₁₀ alkenyl group, a substituted or unsubstituted C₁ to C₁₀ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; or urea, urethane, nitroso, nitro, sulphenyl, sulphonyl, sulfonic acid, or trialkylsilyl. Examples include acetyl, carbobenzyloxy (also benzyloxycarbonyl or carbobenzoxy), formyl, t-butyloxycarbonyl, fluorenylmethyloxycarbonyl, 2-nitrophenylsulfenyl, methanesulfonyl, p-toluenesulfonyl, or p-nitro phenyl sulfonyl group.

According to an embodiment, P² is selected from group comprising of a carboalkoxy group C(═O)Y, wherein Y is OR and wherein R is substituted or unsubstituted C₁ to C₁₀ alkyl group. In an embodiment the P² substitution of compound of formula (III) is selected from, but not limited to, a carboalkoxy group i.e. C(═O)Y, wherein Y is OR and wherein R is substituted or unsubstituted C₁ to C₁₀ alkyl group.

According to an embodiment, the step i) is carried out in presence of a solvent, preferably a biphasic solvent system.

In an embodiment, the biphasic solvent system comprises of water and at least one water-immiscible solvent.

In an embodiment, the water-immiscible solvent is selected from the group comprising of dichloromethane, dichloroethane, chloroform, carbon tetrachloride, diethyl ether, ethyl acetate, hexane, cyclohexane, heptane, methyl-tert-butyl ether toluene and benzene.

According to an embodiment, the step i) is carried out by maintaining the pH in the range of 4 to 7; more preferably in the range of 5 to 6.

According to an embodiment, the pH of step i) is adjusted or maintained using a suitable acid, base or a buffer.

The acid used may be selected from the group including, but not limited to, hydrochloric acid, sulfuric acid, hydrobromic acid, hydroiodic acid, formic acid, and acetic acid.

The base used may be selected from the group including, but not limited to, potassium bicarbonate, potassium hydroxide, potassium carbonate, sodium bicarbonate, sodium hydroxide, sodium carbonate, sodium dihydrogen phosphate, sodium citrate, sodium tartrate, acetanilide, ammonia, theobromine, thiourea, and urea.

In an embodiment, in step i) the compound of formula (II) is treated with ethylchloroformate to obtain a compound of formula (III).

In an embodiment, the step ii) comprises a step of deprotecting the hydroxyl group of the compound of formula (III).

According to another embodiment, step ii) comprises a step of deprotecting hydroxyl group of the compound of formula (III) to obtain compound of formula (IV)

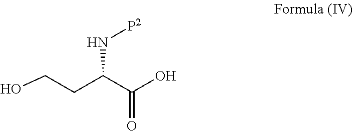

wherein P² is amine protecting group.

In an embodiment, the deprotection of the hydroxyl group of the compound of formula (III) is carried out in presence of an acid. In an embodiment the acid is hydrochloric acid (HCl) or hydrobromic acid (HBr).

In an embodiment, the deprotection of hydroxyl group of the compound of formula (III) is carried out with acid that is generated in-situ in the reaction mixture.

According to an embodiment, the deprotection of hydroxyl group of the compound of formula (III) is carried out in presence of a solvent selected from C₁ to C₅ alcohols.

According to an embodiment, the step ii) of the process may or may not involve isolation of compound of formula (IV).

Preferably, compound of formula (IV) is not isolated.

According to an embodiment, the step ii) of the process further comprises treatment with a halogenating agent.

The halogenating agent used may be selected from the group including, but not limited to, chlorine, phosphorus trichloride (PCl₃), phosphorus pentachloride (PCl₅), phosphorus oxychloride (POCl₃), thionyl chloride, sulfuryl chloride, oxalyl chloride, sulfonyl chloride, acetyl chloride, phosgene, bromine, phosphorus tribromide (PBr₃), boron tribromide (BBr₃), Iodine, phosphorus triiodide (PI₃), N-chlorosuccinimide, and N-bromosuccinimide.

According to another embodiment, in step ii) the halogenation is carried out in presence of a solvent.

The solvent used may be selected from the group including, but not limited to, nitriles like acetonitrile or C₁-C₅ alcohols like methanol, ethanol, isopropanol, n-propanol, butanol, tert-butanol or chlorinated solvent like dichloromethane, dichloroethane, chloroform, 1,1,2-trichloroethane, chlorobenzene, and dichlorobenzene.

In an embodiment, the step ii) comprises treating the compound of formula (III) with a halogenating agent in presence of a solvent selected from C₁ to C₅ alcohols to obtain compound of formula (I); wherein the step of deprotection of hydroxyl group of compound of formula (III) is carried out using an acid generated in-situ in the reaction mixture.

In an embodiment, in the step ii) the compound of formula (III) is converted to compound of formula (I), without requiring isolation of compound of formula (IV)

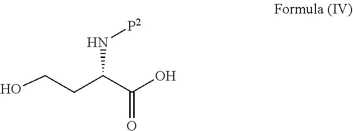

P² is same as defined above.

According to an embodiment, the step i) and ii) are carried out at temperature ranging from −10 to 50° C. for a period in the range from 0.5 to 20 hours.

According to an embodiment, step i) and ii) are performed in one pot synthesis.

According to an embodiment, there is provided a process for preparation of compound of formula (I),

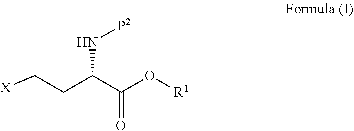

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group;
comprising steps of:
   i) protecting the amine group of compound of formula (II) or its salt to obtain compound of formula (III),

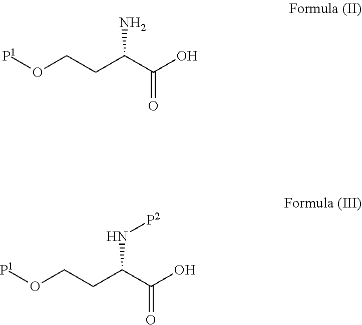

wherein P¹ is hydroxyl-protecting group; and P² is an amino-protecting group; and
ii) converting compound of formula (III) to compound of formula (I),
wherein the step i) is carried out in a biphasic solvent system by maintaining pH in the range of 4 to 7.

According to an embodiment, there is provided a process for preparation of compound of formula (I),

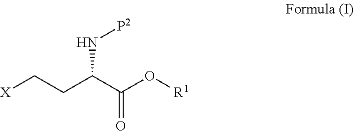

wherein,
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a hydroxyl group;
comprising steps of:
   i) protecting the amine group of a compound of formula (II) or its salt to obtain a compound of formula (III),

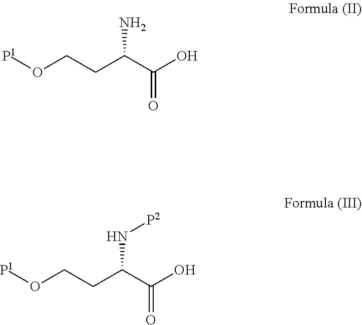

wherein P¹ is hydroxyl-protecting group; and P² is an amino-protecting group; and
ii) converting the compound of formula (III) to a compound of formula (I),
wherein the step i) is carried out in a biphasic solvent system by maintaining pH in the range of 4 to 7.

According to an aspect of the present invention, there is provided a process for preparation of compound of formula (I),

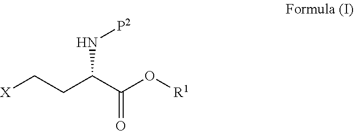

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group, comprising steps of:
   i) protecting the amine group of compound of formula (II) or its salt to obtain compound of formula (III),

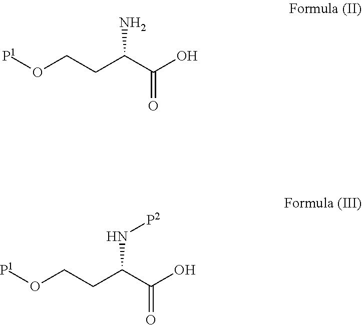

wherein P¹ is hydroxyl-protecting group; and P² is an amino-protecting group; and
ii) converting compound of formula (III) to compound of formula (I), without requiring isolation of compound of formula (IV)

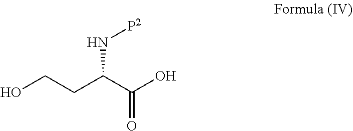

P² is same as defined above;
wherein the step i) is carried out in a biphasic solvent system by maintaining pH in the range of 4 to 7.

According to an embodiment of present invention, the reaction takes route illustrated by scheme I as follows.

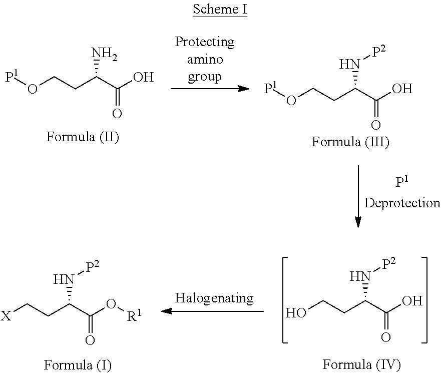

wherein:
P¹ is hydroxyl-protecting group;
P² is amino-protecting group; and
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and X is a halogen.

In an embodiment, the process for the preparation of a compound (I) proceeds via the compound of formula (II).

In an embodiment, the process for the preparation of a compound (I) process via the intermediate of formula (III).

In an embodiment, the process for the preparation of a compound (I) process via the intermediate of formula (IV).

According to an embodiment, compound of formula (IV) may be isolated.

According to another aspect of the present invention, there is provided a process for preparation of compound of formula (I),

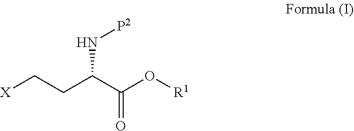

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
comprising steps of:
   a) reacting compound of formula (II) or its salt with an acid to obtain compound of formula (V) or its salt,

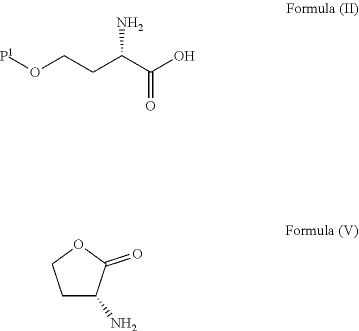

P¹ is hydroxyl-protecting group;
b) protecting the amine group of compound of formula (V) or its salt to obtain compound of formula (VI),

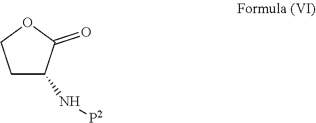

wherein P² is same as defined above; and
c) treating the compound of formula (VI) with a halogenating agent wherein the step b) is carried out by maintaining pH in the range of 4 to 7.

According to an embodiment, the acid used in step a) may be selected from the group comprising of hydrochloric acid, sulfuric acid, hydrobromic acid, hydroiodic acid, formic acid and acetic acid.

In an embodiment, the acid used is hydrobromic acid.

In an embodiment, the step a) of the process may be carried out in an aqueous medium. In an embodiment of present invention, step b) is carried out in a biphasic solvent system.

In an embodiment, the step a) of the process is carried out at temperature ranging from 0° C. to 150° C. and for a period in the range from 1 to 10 hours. Preferably the step a) is carried out at temperature ranging from 20° C. to 80° C.

In an embodiment, the biphasic solvent system comprises of water and at least one water-immiscible solvent. The water-immiscible solvent used may be selected from the group including, but not limited to, dichloromethane, dichloroethane, chloroform, carbon tetrachloride, diethyl ether, ethyl acetate, hexane, cyclohexane, heptane, methyl-tert-butyl ether, toluene, and benzene.

According to an embodiment, the biphasic solvent system comprises of water and dichloromethane.

According to an embodiment, the ratio of water to water-immiscible solvent is in the range of 10:90 to 90:10, preferably in the range of 30:70 to 70:30.

In an embodiment, the step b) is carried out at temperature ranging from −20 to 50° C. In an embodiment, the step b) is carried out at temperature ranging from −20° C. to 20° C., preferably from −5° C. to 10° C.

According to an embodiment of the present invention, the step b) is carried out by maintaining the pH in the range of 4 to 7; more preferably in the range of 5 to 6.

According to an embodiment of the present invention, the pH of step b) is adjusted or maintained using a suitable acid, base or a buffer.

The acid used may be selected from the group including, but not limited to, hydrochloric acid, sulfuric acid, hydrobromic acid, hydroiodic acid, formic acid, and acetic acid.

The base used may be selected from the group including, but not limited to, potassium bicarbonate, potassium hydroxide, potassium carbonate, sodium bicarbonate, sodium hydroxide, sodium carbonate, sodium dihydrogen phosphate, sodium citrate, sodium tartrate, acetanilide, ammonia, theobromine, thiourea, and urea.

The step b) of the process involves an equilibrium reaction system i.e. two reactions are active simultaneously. A forward reaction results in formation of N-protected compound of formula (VI) and at the same time an undesired reversible reaction produces L-homoserine as a by-product. The process of the present invention is skillfully developed in such a way that the side reaction is avoided and the formation of by-product L-homoserine is principally eliminated.

According to another embodiment, in step b) the compound of formula (V) is treated with ethylchloroformate to obtain compound of formula (VI) in presence of a base.

The base used is selected from the group comprising of potassium bicarbonate, potassium hydroxide, potassium carbonate, sodium bicarbonate, sodium hydroxide, sodium carbonate, sodium dihydrogen phosphate, sodium citrate, sodium tartrate, acetanilide, ammonia, theobromine, thiourea and urea.

It was observed that maintaining pH in the range of 4 to 7 in a biphasic solvent system, avoids the formation of by product in great extent.

According to a preferred embodiment of the present invention, the P² substitution of compound of formula (VI) is selected from, but not limited to, a carboalkoxy group.

According to a preferred embodiment of the present invention, in step b) the compound of formula (V) is treated with ethylchloroformate to obtain compound of formula (VI).

According to another embodiment, in step c) the halogenating agent used may be selected from the group including, but not limited to, chlorine, phosphorus trichloride (PCl₃), phosphorus pentachloride (PCl₅), phosphorus oxychloride (POCl₃), thionyl chloride, sulfuryl chloride, oxalyl chloride, sulfonyl chloride, acetyl chloride, phosgene, bromine, phosphorus tribromide (PBr₃), boron tribromide (BBr₃), Iodine, phosphorus triiodide (PI₃), N-chlorosuccinimide, and N-bromosuccinimide.

According to another embodiment, in step c) the halogenating agent used is thionyl chloride.

According to another embodiment of, in step c) of the process is carried out in presence of a suitable solvent.

The solvent used may be selected from the group including, but not limited to, nitriles like acetonitrile or C₁-C₅ alcohols like methanol, ethanol, isopropanol, n-propanol, butanol, tert-butanol or chlorinated solvent like dichloromethane, dichloroethane, chloroform, 1,1,2-trichloroethane, chlorobenzene, and o-dichlorobenzene.

According to an embodiment, the solvent used is selected from C₁-C₅ alcohols.

According to an embodiment, the solvent used is ethanol.

According to an embodiment, the step b) and c) are carried out at temperature ranging from −10 to 50° C. and for a period ranging from 0.5 to 20 hours.

In an embodiment of the present invention, the step a), b) and c) are performed in one pot synthesis.

According to an embodiment, there is provided a process for preparation of compound of formula (I),

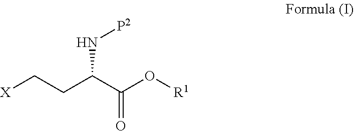

wherein:
P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
comprising steps of:
   a) reacting compound of formula (II) or its salt with an acid to obtain compound of formula (V) or its salt,

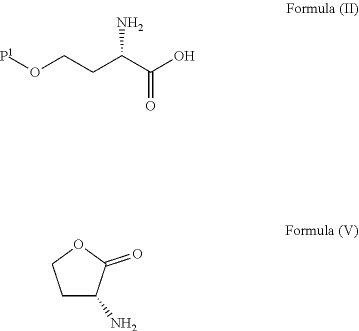

P¹ is hydroxyl-protecting group
b) protecting the amine group of compound of formula (V) or its salt to obtain compound of formula (VI),

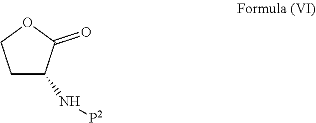

Wherein P² is same as defined above; and
c) treating the compound of formula (VI) with a halogenating agent,
wherein the step b) is carried out in a biphasic solvent system by maintaining pH in the range of 4 to 7.

According to an embodiment, there is provided a process for preparation of compound of formula (I),

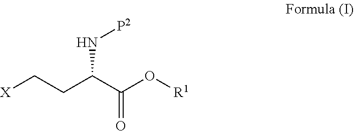

wherein; P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
comprising steps of:
   a) reacting compound of formula (II) or its salt with an acid to obtain compound of formula (V) or its salt, avoiding formation of L-homoserine;

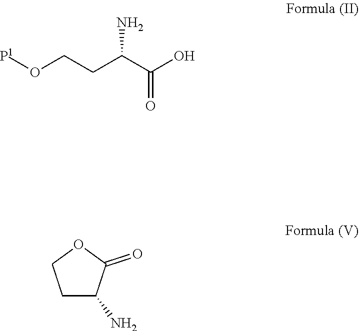

P is hydroxyl-protecting group
b) protecting the amine group of compound of formula (V) or its salt to obtain compound of formula (VI),

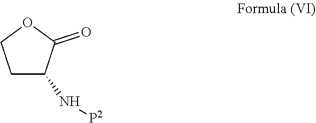

wherein P² is same as defined above; and
c) treating the compound of formula (VI) with a halogenating agent,
wherein the step b) is carried out in a biphasic solvent system by maintaining pH in the range of 4 to 7.

According to an embodiment of present invention, the reaction takes route illustrated by scheme II as follows.

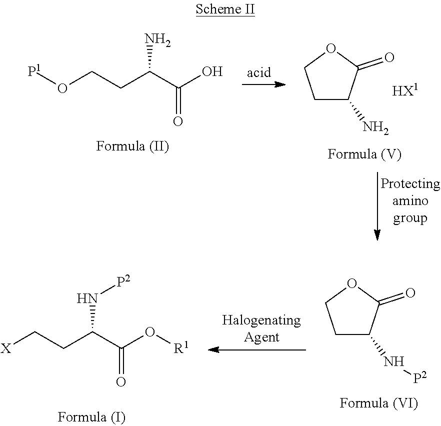

wherein:
P¹ is hydroxyl-protecting group;
P² is amino-protecting group; and
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; X is a halogen or hydroxyl group; HX¹ represent acid addition salt of compound of formula (V).

In an embodiment, the process for the preparation of compound (I) proceeds via the compound of formula (II).

In an embodiment, the process for the preparation of compound (I) proceeds via the intermediate compound of formula (V).

In an embodiment, the process for the preparation of compound (I) proceeds via the intermediate compound of formula (VI).

According to a preferred embodiment, in scheme II, compound of formula (V) is acid addition salt of hydrobromic acid.

According to yet another aspect of the present invention, there is provided a process for preparation of L-glufosinate or its salt, comprising reacting a compound of formula (I) obtained by one of the processes described above, with a phosphorous containing compound.

According to an embodiment, there is provided a process for preparation of L-glufosinate or its salt, comprising reacting a compound of formula (I) obtained by one of the improved processes described above, with a phosphorous containing compound and subsequently converting to L-glufosinate or its salt. In an embodiment the process comprises acid-base treatment to obtain L-glufosinate or its salts.

According to another embodiment, in the process for preparation of L-glufosinate or its salts, the phosphorous containing compound is selected from a trivalent phosphorous-containing compound of formula (VIIa) or a pentavalent phosphorous-containing compound of formula (VIIb),

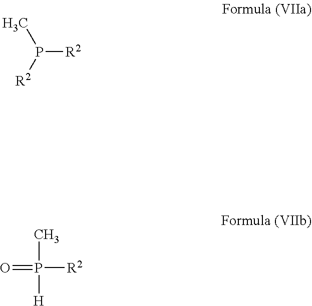

wherein R² is halogen or C₁ to C₆ linear or branched alkoxy group.

According to an embodiment, the process for preparation of L-glufosinate or its salts comprises formation of compound of formula (VIII) by reacting compound of formula (I) and phosphorous containing compound of formula (VIIa) or (VIIb),

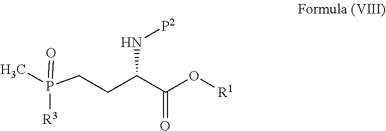

wherein R³ is hydroxyl or C₁ to C₆ linear or branched alkoxy group.

According to a preferred embodiment, the phosphorous containing compound used is selected from diethyl methylphosphonite, methyl dichlorophosphine and methyl isobutyl phosphinate.

According to a preferred embodiment, there is provided a process for preparation of L-glufosinate or its salt, comprising reacting a compound of formula (I) wherein X is a halogen, with a phosphorous containing compound of formula (VIIa) wherein R² is C₁ to C₆ linear or branched alkoxy group and subsequently converting the product into L-glufosinate or its salt.

According to an embodiment, the compound of formula (I) wherein X is a halogen, is reacted with a phosphorous containing compound of formula (VIIa) at temperature ranging from 50 to 200° C.

According to a preferred embodiment, there is provided a process for preparation of L-glufosinate or its salt, comprising reacting a compound of formula (I) wherein X is hydroxyl group, with a phosphorous containing compound of formula (VIIa) wherein R² is halogen and subsequently converting the reaction product into L-glufosinate or its salt.

According to a preferred embodiment, there is provided a process for preparation of L-glufosinate or its salt, comprising reacting a compound of formula (I) wherein X is a halogen, with a phosphorous containing compound of formula (VIIb) wherein R² is C₁ to C₆ linear or branched alkoxy group and subsequently converting the reaction product into L-glufosinate or its salt.

According to a preferred embodiment, the compound of formula VIII is converted to L-glufosinate or its salt by subjecting it to acid-base treatment.

According to an embodiment, acid treatment is carried out using acids such as hydrochloric acid, sulfuric acid, hydrobromic acid or hydroiodic acid.

According to an embodiment, base treatment is carried out using an organic or inorganic base. Base used may be selected from group consisting of, but not limited to, potassium bicarbonate, potassium hydroxide, potassium carbonate, sodium bicarbonate, sodium hydroxide, sodium carbonate, sodium dihydrogen phosphate, sodium citrate, sodium tartrate, acetanilide, ammonia, amines, theobromine, thiourea, and urea.

According to embodiments of present invention, the L-glufosinate salt such as monosodium salt, disodium salt, monopotassium salt, dipotassium salt, calcium salt, ammonium salt, —NH₃(CH₃)⁺ salt, —NH₂(CH₃)₂⁺ salt, —NH(CH₃)₃⁺ salt, —NH(CH₃)₂(C₂H₄OH)⁺ salt, —NH₂(CH₃)(C₂H₄OH)⁺ salt or the likes, can be prepared.

According to an embodiment, L-glufosinate or its salt is synthesized from compound of formula (II) or its salt, without isolating compound of formula (I) in one pot synthesis.

According to an embodiment of present invention, the process for preparation of L-gluosinate or its salt from compound of formula (I) takes route illustrated by scheme III as follows.

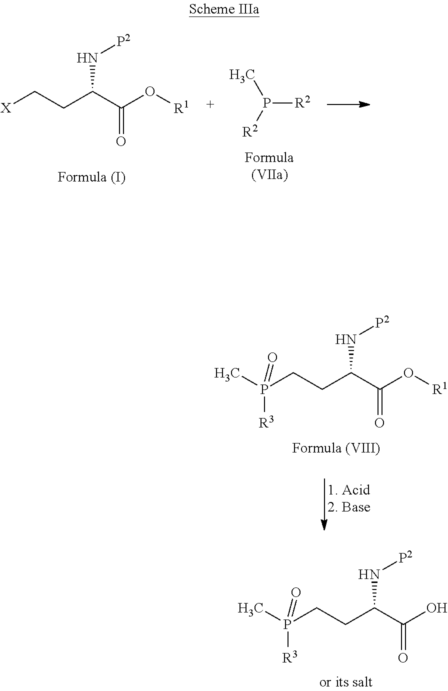

wherein:
P² is amino-protecting group; and
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; R² is C₁ to C₆ linear or branched alkoxy group, R³ is hydroxyl or C₁ to C₆ linear or branched alkoxy group and X is a halogen.

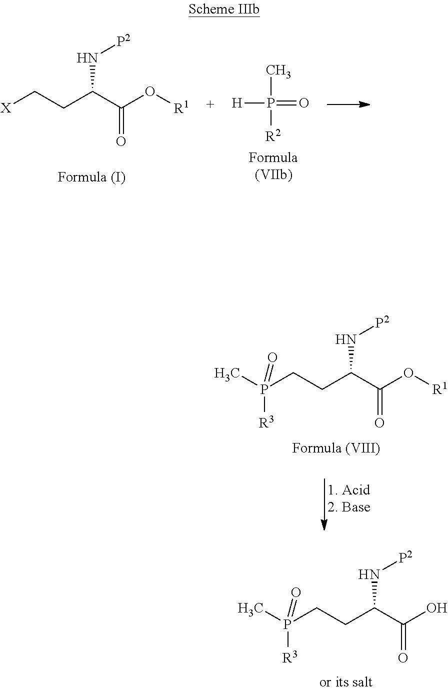

wherein, P², R¹, R² and X are same as defined above.

According to an aspect of the present invention, there is provided a compound of formula (III)

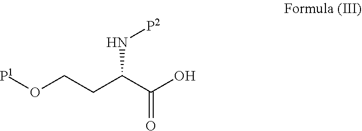

wherein P¹ and P² are defined above.

According to an embodiment, there is provided a compound of formula (IIIa)

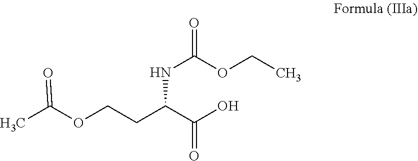 [SMILES: CCOC(=O)N[C@@H](CCOC(C)=O)C(=O)O]

In an embodiment there is provided a process for preparation of compound of formula (III) comprising protecting an amine group of compounds of formula (II) or its salt to obtain compound of formula (III),

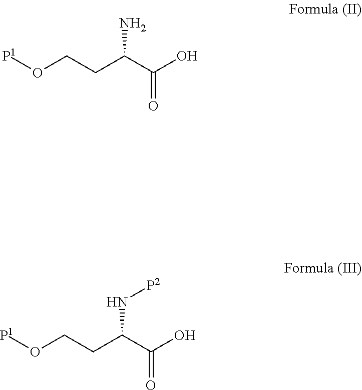

wherein, P¹ is hydroxyl-protecting group; and P² is an amino-protecting group.

In an embodiment the invention provides use of a compound of formula (IIIa) for synthesis of L-glufosinate or its salts.

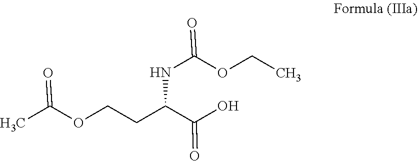 [SMILES: CCOC(=O)N[C@@H](CCOC(C)=O)C(=O)O]

In an embodiment, there is provided a process for the preparation of L-glufosinate or its salt, wherein the process proceeds via an intermediate of formula (IIIa).

In an embodiment, there is provided a process for the preparation of L-glufosinate or its salt, wherein the process proceeds via a compound of formula (I).

In another aspect, there is provided a process for the preparation of L-glufosinate or its salt, wherein the process proceeds via an intermediate of formula (VIII).

In another embodiment there is provided use of L-glufosinate or its salts prepared according to the present process for the preparation of agrochemical composition or formulation.

In another embodiment there is provided use of L-glufosinate or its salts prepared using the compound of formula III in one pot synthesis for the preparation of agrochemical composition or formulation.

In an embodiment, the agrochemical composition comprising L-glufosinate or its salts prepared according to the present process as described herein.

According to another embodiment, the present invention provides a herbicidal composition comprising L-glufosinate or its salts prepared according to the process as described herein and an agrochemically acceptable excipients.

In an embodiment the agrochemical composition comprising L-glufosinate or L-glufosinate ammonium prepared according to the present invention from 1 to 99% by weight of the total composition and an agrochemically acceptable excipient from about 1 to 50% by weight of the total composition.

In an embodiment, the agronomically acceptable excipients can be selected from, but not limited to, surfactants, solvent, fertilizer, pH modifiers, crystallization inhibitors, viscosity modifiers, suspending agents, spray droplet modifiers, pigments, antioxidants, foaming agents, light-blocking agents, compatibilizing agents, antifoam agents, sequestering agents, neutralizing agents, corrosion inhibitors, dyes, odorants, spreading agents, penetration aids, micronutrients, emollients, lubricants, sticking agents, dispersing agents, thickening agents, freezing point depressants, antimicrobial agents, and the like.

According to an embodiment of present invention, the dispersion comprising single isomer of glufosinate salt and at least one organic solvent may further comprise a surfactant.

The surfactants used in the process may be selected from anionic, cationic or zwitterionic and/or non-ionic surface-active compounds (surfactants) or combinations thereof.

Examples of anionic surfactants include: anionic derivatives of fatty alcohols having 10-24 carbon atoms in the form of ether carboxylates, sulfonates, sulfates, and phosphates, and their inorganic salts (e.g., alkali metal and alkaline earth metal salts) and organic salts (e.g., salts based on amine or alkanolamine); anionic derivatives of copolymers consisting of EO (ethylene oxide), PO (propylene oxide) and/or BO (butylene oxide) units, in the form of ether carboxylates, sulfonates, sulfates, and phosphates, and their inorganic salts (e.g., alkali metal and alkaline earth metal salts) and organic salts (e.g., salts based on amine or alkanolamine); derivatives of alkylene oxide adducts of alcohols, in the form of ether carboxylates, sulfonates, sulfates and phosphates, and their inorganic salts (e.g., alkali metal and alkaline earth metal salts) and organic salts (e.g., salts based on amine or alkanolamine); derivatives of fatty acid alkoxylates, in the form of ether carboxylates, sulfonates, sulfates and phosphates, and their inorganic salts (e.g., alkali metal and alkaline earth metal salts) and organic salts (e.g., salts based on amine or alkanolamine); alkyl ether phosphate, sulfosuccinate & its derivatives, sulfosuccinate half ester, alkyl sulfosuccinate mono ester and diester salts.

Examples of cationic or zwitterionic surfactants may be selected from alkylene oxide adducts of fatty amines, quaternary ammonium compounds having 8 to 22 carbon atoms (C₈-C₂₂), surface-active zwitterionic compounds such as taurides, betaines and sulfobetaines.

Examples of non-ionic surfactants are: alkylpolyglycosides, alkyl glucamide, alkyl amine oxides having C₈ to C₂₀ carbon atoms, alcohol ethoxylate, fatty acid methyl ester, sorbitan ester and ethoxylated sorbitan ester, ethoxylated alkylphenol, ethoxylated tristyrylphenol and alkyl amide, fatty alcohols having 10-24 carbon atoms with 0-60 ethylene oxide (EO) and/or 0-20 propylene oxide (PO) and/or 0-15 butylene oxide (BO) in any order; fatty acid alkoxylates and triglyceride alkoxylates; fatty acid amide alkoxylates; alkylene oxide adducts of alkynediols; sugar derivatives such as amino sugars and amido sugars; polyacrylic and polymethacrylic derivatives; polyamides such as modified gelatins or derivatized polyaspartic acid; surfactant polyvinyl compounds such as modified PVP; polyol-based alkylene oxide adducts; polyglycerides and derivatives thereof.

In an aspect there is provided use of the present composition prepared according to the present invention to control harmful/undesired plants.

In an embodiment the present invention provides use of present composition comprising L-glufosinate or its salts prepared according to the present invention optionally with other auxiliary ingredients to control harmful/undesired plants/weeds.

The above-mentioned compositions provide effective weed control to keep agricultural crops free from undesired competing plants and thus to safeguard and/or increase the yields.

In another embodiment the present invention provides a method of controlling undesired plants by applying the present compositions comprising L-glufosinate or L-glufosinate ammonium prepared according to the present invention.

In an embodiment, the composition of the present invention may be applied to the locus either simultaneously or sequentially, such that the herbicide may be applied in a tank mix or as a pre-mixed composition.

In an embodiment, the method comprises pre or post emergent application of present compositions.

The present method may be carried out by spraying the suggested tank mixes or may be formulated as a kit-of-parts containing various components that may be mixed as instructed prior to spraying.

According to an embodiment of the present invention, there is provided a kit comprising the present composition containing L-glufosinate or its salt prepared by present invention for controlling harmful plants/weeds.

### Advantages of the Present Invention

1. The present invention provides a shorter, economical and scalable route for synthesis of L-glufosinate and compound of formula (I)
2. The present invention provides an alternative route for preparation of compound of formula (I)
3. The present invention provides one pot processes for preparation of compound of formula (I)
4. The present invention provides a compound of formula (I) and its intermediates in high purity.

The process for preparation of compound of formula (I) and L-glufosinate or its salt according to the invention is ascertained by the experiments as exemplified below.

These examples are merely illustrations and are not to be understood as limiting the scope and underlying principles of the invention in any way. Indeed, various modifications of the invention in addition to those shown and described herein will become apparent to those skilled in the art from the following examples and foregoing description.

### EXAMPLES

### Methods

The qualitative analysis of L-isomer and D-isomer in L-glufosinate ammonium is performed using HPLC Column—Chirex 3126 (D)-penicillamine LC column (150×4.6 mm).

The qualitative analysis of Formula IIIa is performed by using HPLC Column—Inertsil—C₁₈ (250×4.6 mm i.d., 5 micron).

### Example 1

### Process for Preparation of Ethyl 4-chloro-2-[(methoxy carbonyl)amino]butanoate (Formula I)

### Step 1: Preparation of L-homoserine lactone salt (Formula V) by Cyclisation of O-acetyl-L-homoserine

25 g of O-acetyl-L-Homoserine was added to 80 g (3 equiv.) of 48% aqueous hydrogen bromide (3 equiv.) solution at room temperature. The reaction mixture was heated to 55° C. and maintained for 4 hours. After completion of the reaction, excess of HBr and water were removed by distillation to get crude L-homoserine lactone salt. The crude product was the triturated with 50 ml isopropanol, filtered and dried to obtain L-homoserine lactone hydrobromide salt having purity of about 98%.

### Step 2: Preparation of N-carboethoxy L-Homoserine lactone (Formula VI)

25 g L-Homoserine lactone hydrobromide from step-1, was added to 250 ml of pre-cooled solvent system comprising water and dichloromethane (50:50) at 0-5° C. The mixture was stirred followed by simultaneous addition of 15.6 g (1.05 equiv.) of ethyl chloroformate and 17.3 g (1.2 equiv.) 50% sodium carbonate aqueous solution, maintaining the pH between 5 to 6 and temperature below 5° C. within 1 hour. After completion of the reaction, the organic layer was separated, and the solvent was distilled off to yield N-carboethoxy-L-Homoserine lactone product having purity of about 98%.

### Step 3: Preparation of Ethyl 4-chloro-2-[(methoxy carbonyl)amino] butanoate (Formula I)

21.2 g of N-carboethoxy-L-Homoserine lactone from step 2, was added to 100 ml ethanol at room temperature followed by dropwise addition of 30.6 g (2.1 equiv.) of thionyl chloride. The reaction mixture was stirred at room temperature for 4 to 5 hours. After completion of reaction, the volatiles were removed by distillation and the product thus obtained was washed with 50 ml of water and dried to yield 27.6 g of product having purity of about 95%.

### Example 2: Preparation of L-glufosinate ammonium

10.0 gm of ethyl 4-chloro-2-[(methoxy carbonyl)amino] butanoate (formula I) prepared from step 3 of example 1 and 6.9 g (1.5 equiv.) of diethyl methylphosphonite were charged to a reaction flask at 25-30° C. The mixture was heated at 140° C. for 20 hours while flushing with nitrogen continuously in the system. After completion of the reaction, the excess diethyl methylphosphonite was distilled off under vacuum to get 12 g of ethyl (2S)-2-[(methoxy carbonyl)amino]-4-[ethoxy(methyl)phosphoryl] butanoate 40.0 gm of conc. HCl (10 equiv.) was added to the flask and refluxed the mixture for 16 hrs. After the completion of the reaction, water was distilled off completely. Then 62.0 ml of methanol was added and purged with dry ammonia gas till pH 8-8.5. The reaction mixture was heated at 60° C. for 4 hrs. The mixture was then cooled, the precipitate obtained was filtered and washed with 6 ml methanol and dried under vacuum at 50° C. to get L-glufosinate ammonium (% w/w purity more than 96%, L:D ratio-97:03).

### Example 3: Preparation of Ethyl 4-chloro-2-[(methoxy carbonyl)amino]butanoate (Formula I) Through Compound of Formula V

25 g of O-acetyl-L-Homoserine was added to 80 g of 48% aqueous hydrogen bromide solution at room temperature. The reaction mixture was heated to 55° C. and maintained for 4 hours. After completion of the reaction, excess of HBr and water were removed by distillation to get crude L-homoserine lactone hydrobromide salt. To the crude L-Homoserine lactone hydrobromide salt was added to 250 ml of pre-cooled solvent system comprising water and dichloromethane (50:50) at 0-5° C. The mixture was stirred followed by simultaneous addition of 15.6 g (1.05 equiv.) of ethyl chloroformate and 17.3 g (1.2 equiv.) 50% sodium carbonate aqueous solution, maintaining the pH between 5 to 6 and temperature below 5° C. within 1 hour. After completion of the reaction, the organic layer was separated, and the solvent was distilled off to half volume to get mixture comprising N-carboethoxy-L-Homoserine lactone. To said mixture was added to 100 ml ethanol at room temperature followed by dropwise addition of 30.6 g (2.1 equiv.) of thionyl chloride. The reaction mixture was stirred at room temperature for 4 to 5 hours. After completion of reaction, the volatiles were removed by distillation and the product thus obtained was washed with 50 ml of water and dried to yield product having purity of about 95%.

### Example 4: Preparation of N-carboethoxy-O-acetyl-L-Homoserine (Formula IIIa)

25 g of O-acetyl-L-Homoserine was added to 250 ml was added to 250 ml of pre-cooled solvent system comprising water and dichloromethane (50:50) at 0-5° C. The mixture was stirred followed by simultaneous addition of 18.45 g (1.1 equiv.) of ethyl chloroformate and 19.75 g (1.2 equiv.) 50% sodium carbonate aqueous solution, maintaining the pH between 4 to 6 and temperature below 0° C. within 1 hour. After completion of the reaction, the organic layer was separated, and the solvent was distilled off to yield N-carboethoxy-O-acetyl-L-Homoserine.

LC-MS (m/z): 233 (M−H)⁻

### Example 5: Preparation of Ethyl 4-chloro-2-[(methoxy carbonyl)amino]butanoate (Formula I) Through Compound of Formula IIIa

100 g of O-acetyl-L-Homoserine was added to 1000 ml of pre-cooled solvent system comprising water and dichloromethane (50:50) at 0-5° C. The mixture was stirred followed by simultaneous addition of 70.7 g (1.05 equiv.) of ethyl chloroformate and 79 g of 50% sodium carbonate aqueous solution, maintaining the pH between 5 to 6 and temperature below −5° C. within 1 hour. After completion of the reaction, the organic layer was separated, and the solvent was distilled off to half volume. To the mixture was then added 400 ml of ethanol and 185 g of thionyl chloride at room temperature. After completion of reaction, the volatiles were removed by distillation and the brown liquid obtained was washed with 100 ml of water and dried to yield product having purity of about 95%.

1. A process for preparation of compound of formula (I), 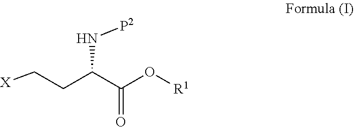
wherein P² is an amino-protecting group;
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
comprising steps of,
i) protecting an amine group of compound of formula (II) or its salt to obtain compound of formula (III), 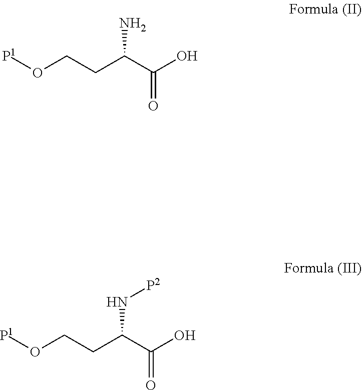
wherein, P¹ is hydroxyl-protecting group; and P² is an amino-protecting group; and
ii) converting compound of formula (III) to compound of formula (I)
wherein the step i) is carried out by maintaining pH in the range of 4 to 7.

2. The process as claimed in claim 1, wherein P¹ is selected from the group comprising of an acyl group derived from substituted or unsubstituted C₁ to C₁₅ mono or dicarboxylic acid.

3. The process as claimed in claim 1, wherein P² is selected from group comprising of a carboalkoxy group C(═O)Y, wherein Y is OR and wherein R is substituted or unsubstituted C₁ to C₁₀ alkyl group.

4. The process as claimed in claim 1, wherein the step i) is carried out in presence of a biphasic solvent system.

5. The process as claimed in claim 4, wherein said biphasic solvent system comprises of water and at least one water-immiscible solvent.

6. The process as claimed in claim 5, wherein said water-immiscible solvent is selected from the group comprising of dichloromethane, dichloroethane, chloroform, carbon tetrachloride, diethyl ether, ethyl acetate, hexane, cyclohexane, heptane, methyl-tert-butyl ether toluene and benzene.

7. The process as claimed in claim 1, wherein the step ii) comprises a step of deprotecting the hydroxyl group of the compound of formula (III).

8. The process as claimed in claim 7, wherein said deprotection is carried out in presence of an acid.

9. The process as claimed in claim 8, wherein said acid generated in-situ in the reaction mixture.

10. The process as claimed in claim 7, wherein said deprotection is carried out in presence of a solvent selected from C₁ to C₅ alcohols.

11. The process as claimed in claim 7, wherein the step ii) of the process further comprises treatment with a halogenating agent.

12. The process as claimed in claim 1, wherein the step ii) comprises treating the compound of formula (III) with a halogenating agent in presence of a solvent selected from C₁ to C₅ alcohols to obtain compound of formula (I); wherein the step of deprotection of hydroxyl group of compound of formula (III) is carried out using an acid generated in-situ in the reaction mixture.

13. The process as claimed in claim 11, wherein said halogenating agent is selected from the group comprising of chlorine, phosphorus trichloride, phosphorus pentachloride, phosphorus oxychloride, thionyl chloride, sulfuryl chloride, oxalyl chloride, sulfonyl chloride, acetyl chloride, phosgene, bromine, phosphorus tribromide, boron tribromide, Iodine, phosphorus triiodide, N-chlorosuccinimide, and N-bromosuccinimide.

14. The process as claimed in claim 1, wherein the step i) and ii) are carried out at temperature ranging from −10 to 50° C.

15. The process as claimed in claim 1, wherein the step i) and ii) are performed in one pot synthesis.

16. The process as claimed in claim 1, wherein in the step ii) the compound of formula (III) is converted to compound of formula (I), without requiring isolation of compound of formula (IV) 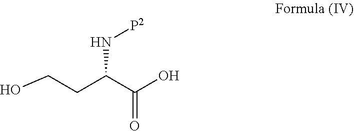
P² is same as defined above.

17. A process for preparation of compound of formula (I), 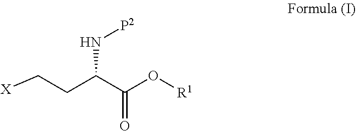
wherein P² is an amino-protecting group
R¹ is hydrogen, substituted or unsubstituted C₁ to C₆ alkyl group, a substituted or unsubstituted C₁ to C₆ alkenyl group, a substituted or unsubstituted C₁ to C₆ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; and
X is a halogen or hydroxyl group,
comprising steps of,
a) reacting a compound of formula (II) or its salts with an acid to obtain compound of formula (V) or its salt, 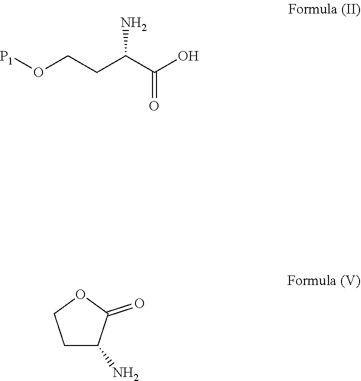
P¹ is hydroxyl-protecting group
b) protecting the amine group of compound of formula (V) or its salt to obtain compound of formula (VI), 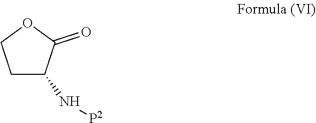
wherein P² is same as defined above;
c) treating the compound of formula (VI) with a halogenating agent
wherein the step b) is carried out by maintaining pH in the range of 4 to 7;
wherein said acid used in step a) is selected from hydrobromic acid and hydroiodic acid.

18. The process as claimed in claim 17, wherein said step a) is carried out in an aqueous medium and step b) is carried out in a biphasic solvent system.

19. The process as claimed in claim 18, wherein said biphasic solvent system comprises of water and at least one water-immiscible solvent.

20. The process as claimed in claim 17, wherein said step a) is carried out at temperature ranging from 0° C. to 150° C.

21. The process as claimed in claim 17, wherein said step b) and c) are carried out at temperature ranging from −10 to 50° C.

22. The process as claimed in claim 17, wherein said step a), b) and c) are performed in one pot synthesis.

23. A process for preparation of L-glufosinate or its salt, comprising reacting a compound of formula (I) with a phosphorous containing compound and subsequently converting to L-glufosinate or its salt, wherein compound of formula (I) is prepared by process as claimed in claim 1.

24. The process as claimed in claim 23, wherein the phosphorous containing compound is selected from diethyl methylphosphonite, methyl dichlorophosphine and methyl isobutyl phosphinate.

25. A compound of formula (IIIa)

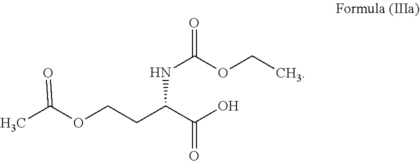 [SMILES: CCOC(=O)N[C@@H](CCOC(C)=O)C(=O)O]

26. A process for preparation of compound of formula (III) comprising protecting an amine group of compounds of formula (II) or its salt to obtain compound of formula (III), 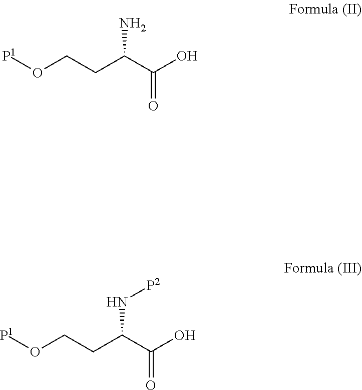
wherein, P¹ is hydroxyl-protecting group; and P² is an amino-protecting group.

27. Use of a compound of formula IIIa for synthesis of L-glufosinate or its salts.

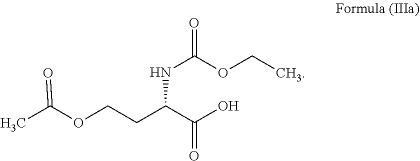 [SMILES: CCOC(=O)N[C@@H](CCOC(C)=O)C(=O)O]

28. The process as claimed in claim 12, wherein said halogenating agent is selected from the group comprising of chlorine, phosphorus trichloride, phosphorus pentachloride, phosphorus oxychloride, thionyl chloride, sulfuryl chloride, oxalyl chloride, sulfonyl chloride, acetyl chloride, phosgene, bromine, phosphorus tribromide, boron tribromide, Iodine, phosphorus triiodide, N-chlorosuccinimide, and N-bromosuccinimide.

29. A process for preparation of L-glufosinate or its salt, comprising reacting a compound of formula (I) with a phosphorous containing compound and subsequently converting to L-glufosinate or its salt, wherein compound of formula (I) is prepared by process as claimed in claim 17.

30. The process as claimed in claim 29, wherein the phosphorous containing compound is selected from diethyl methylphosphonite, methyl dichlorophosphine and methyl isobutyl phosphinate.
